(19) United States
(12) Patent Application Publication
(10) Pub. No.: US 2025/0210144 A1
(43) Pub. Date: Jun. 26, 2025

(54) BASE SEQUENCE ANALYSIS METHOD AND GENE ANALYZER

(71) Applicant: Hitachi High-Tech Corporation, Tokyo (JP)

(72) Inventors: Toru YOKOYAMA, Tokyo (JP); Motohiro YAMAZAKI, Tokyo (JP); Hirokazu KATO, Tokyo (JP); Noriyuki SUMIDA, Tokyo (JP)

(21) Appl. No.: 18/852,114

(22) PCT Filed: Apr. 5, 2022
(86) PCT No.: PCT/JP2022/017118

§ 371 Date: Sep. 27, 2024

(51) Int. Cl. G16B 40/10; C12Q 1/6869; G01N 21/64; G01N 27/447

(52) U.S. Cl. CPC G16B 40/10; C12Q 1/6869; G01N 21/6486; G01N 27/447

(57) ABSTRACT

Provided is a gene analyzer configured to: acquire electrophoresis data which is time-series data of signal intensities of a plurality of frequencies acquired by subjecting a sample to electrophoresis; identify a single fluorescence spectrum time at which a single fluorescence spectrum which is a spectrum derived from only one base is present, by using the electrophoresis data; calculate a spectral shift model by using a spectrum obtained from a color conversion matrix at the single fluorescence spectrum time and the single fluorescence spectrum at the single fluorescence spectrum time; correct the electrophoresis data by using the spectral shift model; and identify a base sequence of the sample by using the corrected electrophoresis data.

### BACKGROUND OF THE INVENTION

This invention relates to a gene analyzer for analyzing a base sequence of a sample by using electrophoresis and a method therefor.

A capillary electrophoresis device acquires electrophoresis data by irradiating excitation light while separating DNA fragments by electrophoresis. The excitation light has a predetermined width, and it is thus known that pull-ups (quasi peaks) due to spectral shift occur (see, for example, paragraphs 0050 and 0051 of JP 2006-292368 A). The accuracy of base sequence analysis is reduced by the pull-ups.

### CITATION LIST

### Patent Literature

PTL 1: JP 2006-292368 A
PTL 2: JP 2014-117222 A
PTL 3: JP 2020-41876 A

### SUMMARY OF THE INVENTION

In JP 2006-292368 A, there is disclosed an optical detection unit which reduces pull-ups. However, spectral shift varies depending on an error in the optical system of each device and differences in environment such as electrophoresis voltage and temperature, and thus it is difficult to model spectral shift precisely. This invention implements a method for reducing pull-ups through information processing.

A typical example of the invention disclosed in this application is as follows. Specifically, there is provided a base sequence analysis method, which is executed by a gene analyzer configured to analyze a base sequence of a sample, the base sequence analysis method including: a first step of acquiring, by the gene analyzer, electrophoresis data which is time-series data of signal intensities of a plurality of frequencies acquired by subjecting the sample to electrophoresis; a second step of identifying, by the gene analyzer, a single fluorescence spectrum time at which a single fluorescence spectrum which is a spectrum derived from only one base is present, by using the electrophoresis data; a third step of calculating, by the gene analyzer, a spectral shift model by using a spectrum obtained from a color conversion matrix at the single fluorescence spectrum time and the single fluorescence spectrum at the single fluorescence spectrum time; a fourth step of correcting, by the gene analyzer, the electrophoresis data by using the spectral shift model; and a fifth step of identifying, by the gene analyzer, a base sequence of the sample by using the corrected electrophoresis data.

According to this invention, it is possible to reduce pull-ups through information processing and improve the accuracy of base sequence analysis by regarding a single fluorescence spectrum as a shifted spectrum obtained from a color conversion matrix. Problems, configurations, and effects other than those described above become apparent through the following description of embodiments.

### BRIEF DESCRIPTION OF THE DRAWINGS

The present invention can be appreciated by the description which follows in conjunction with the following figures, wherein:

FIG. 1 is a diagram for illustrating a configuration example of a gene analyzer 100 of a first embodiment of this invention;

FIG. 2 is a diagram for illustrating an example of a configuration of the electrophoresis device 110 in the first embodiment;

FIG. 3 is a flowchart for illustrating an outline of processing executed by the gene analyzer 100 of the first embodiment;

FIG. 4 is a flowchart for illustrating electrophoresis processing executed by the electrophoresis device 110 in the first embodiment;

FIG. 5 is a flowchart for illustrating spectral correction processing executed by the data analyzer 111 in the first embodiment;

FIG. 6 is a flowchart for illustrating single fluorescence spectrum correction processing executed by the data analyzer 111 in the first embodiment;

FIG. 7 is a diagram for illustrating images of the single fluorescence spectrum correction processing in the first embodiment;

FIG. 8 is a flowchart for illustrating non-single fluorescence spectrum correction processing executed by the data analyzer 111 in the first embodiment; and

FIG. 9 is a diagram for illustrating images of the non-single fluorescence spectrum correction processing in the first embodiment.

### DETAILED DESCRIPTION OF THE PREFERRED EMBODIMENTS

Now, description is given of at least one embodiment of this invention referring to the drawings. It should be noted that this invention is not to be construed by limiting the invention to the content described in the following at least one embodiment. A person skilled in the art would easily recognize that specific configurations described in the following at least one embodiment may be changed within the scope of the concept and the gist of this invention.

In configurations of the at least one embodiment of this invention described below, the same or similar components or functions are denoted by the same reference numerals, and a redundant description thereof is omitted here.

Notations of, for example, “first”, “second”, and “third” herein are assigned to distinguish between components, and do not necessarily limit the number or order of those components.

The position, size, shape, range, and others of each component illustrated in, for example, the drawings may not represent the actual position, size, shape, range, and others in order to facilitate understanding of this invention. Thus, this invention is not limited to the position, size, shape, range, and others disclosed in, for example, the drawings.

### First Embodiment

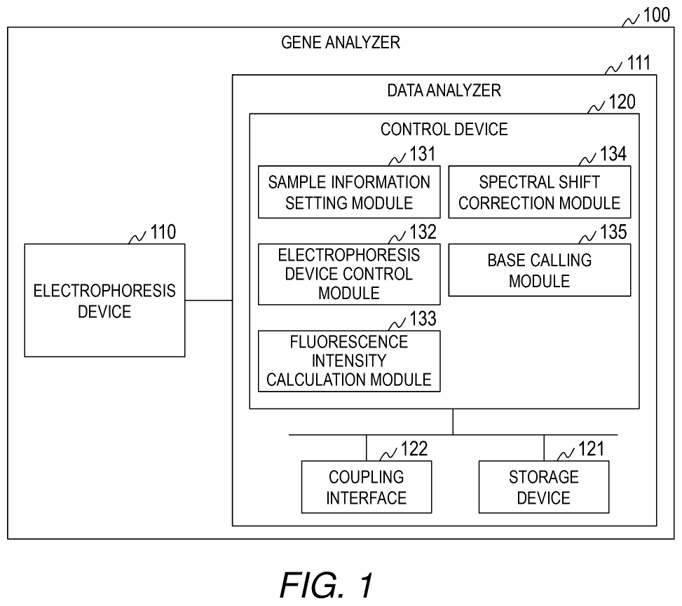

FIG. 1 is a diagram for illustrating a configuration example of a gene analyzer 100 of a first embodiment of this invention.

The gene analyzer 100 includes an electrophoresis device 110 and a data analyzer 111. The electrophoresis device 110 and the data analyzer 111 are communicably coupled by using a communication cable.

The data analyzer 111 includes a control device 120, a storage device 121, and a coupling interface 122.

The control device 120 controls the electrophoresis device 110 and processes data. The control device 120 is, for example, a central processing unit (CPU) or a graphics processing unit (GPU).

The storage device 121 stores, for example, programs executed by the control device 120, setting information on the electrophoresis device 110, and information used in various processes. The storage device 121 is, for example, a memory.

The coupling interface 122 is an interface which is coupled to an input device and an output device, or an interface which is coupled to an external device via a network. The data analyzer 111 presents information to the user and receives information input by the user via the coupling interface 122.

The control device 120 operates as a sample information setting module 131, an electrophoresis device control module 132, a fluorescence intensity calculation module 133, a spectral shift correction module 134, and a base calling module 135 by executing programs stored in the storage device 121. In the following description, when processing is described by using a functional module as the subject of the sentence, this means that the control device 120 is executing a program.

The electrophoresis device 110 performs electrophoresis on a sample (DNA fragment) and acquires electrophoresis data. The electrophoresis data is time-series data of signal intensities (brightness values) at a plurality of frequencies.

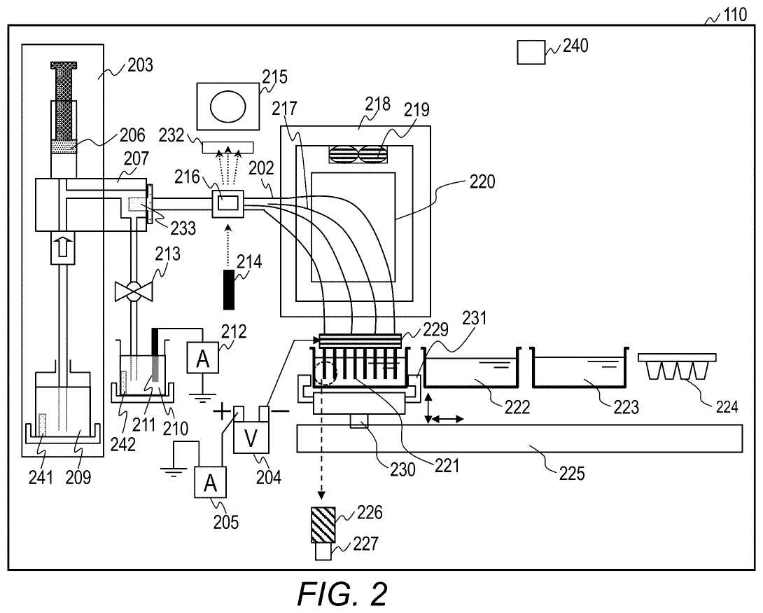

The configuration of the electrophoresis device 110 is now described. FIG. 2 is a diagram for illustrating an example of a configuration of the electrophoresis device 110 in the first embodiment.

The electrophoresis device 110 includes a detection unit 216, a thermostatic bath 218, a conveyance device 225, a high-voltage power source 204, a first ammeter 205, an anode electrode 211, a second ammeter 212, a capillary array 217, and a pump mechanism 203.

The capillary array 217 is a replaceable member including a plurality of (for example, eight) capillaries 202, and includes a load header 229, the detection unit 216, and a capillary head 233. When the capillaries 202 become damaged or deteriorate in quality, the capillary array 217 can be replaced with a new capillary array 217.

Each capillary 202 is made of a glass tube having an inner diameter of several tens to several hundred microns and an outer diameter of several hundred microns, and the surface of the capillary 202 is coated with polyimide to improve strength. However, a light irradiation portion on which laser light is irradiated has a structure in which the polyimide coating is removed so that internally emitted light easily escapes to the outside. The inside of the capillaries 202 is filled with a separation medium to give a difference in migration speed during electrophoresis. The separation medium may be any one of a fluid medium and a non-fluid medium, but in the first embodiment, a fluid polymer is used.

The high-voltage power source 204 applies a high voltage to the capillaries 202. The first ammeter 205 detects the current emitted from the high-voltage power source 204. The second ammeter 212 detects the current flowing through the anode electrode 211.

An optical detection unit which detects information light obtained from the sample includes a light source 214 which irradiates excitation light on the detection unit 216, an optical detector 215 which detects light emitted in the detection unit 216, and a diffraction grating 232. The detection unit 216 is a member which acquires sample-dependent information.

When the sample in a capillary 202 separated by electrophoresis is to be detected, excitation light from the light source 214 is irradiated onto the detection unit 216, thereby generating fluorescence having a wavelength dependent on the sample as information light. Further, the diffraction grating 232 disperses the information light in the wavelength direction, and the optical detector 215 detects the dispersed information light and analyzes the sample.

Each capillary cathode end 227 is fixed through a metal hollow electrode 226, and the tip of the capillary 202 protrudes from the hollow electrodes 226 by about 0.5 millimeters. Further, all the hollow electrodes 226 arranged for the capillaries 202 are attached to the load header 229 as one unit. In addition, all the hollow electrodes 226 are electrically connected to the high-voltage power source 204 mounted on the main body of the device, and function as cathode electrodes when a voltage is required to be applied, such as during electrophoresis and sample introduction.

The capillary ends (other ends) opposite to the capillary cathode ends 227 are bundled together by the capillary head 233. The capillary head 233 can be connected to a block 207 in a pressure-tight manner. The high voltage from the high-voltage power source 204 is applied between the load header 229 and the capillary head 233. New polymer is then filled into the capillaries 202 from the other end through use of a syringe 206. The refilling of polymer into the capillaries 202 is performed after each measurement to improve the measurement performance.

The pump mechanism 203 includes the syringe 206 and a mechanism system for pressurizing the syringe 206, and injects the polymer into the capillaries 202.

The block 207 is a connection part for allowing the syringe 206, the capillary array 217, an anode buffer container 210, and a polymer container 209 to communicate with each other.

The thermostatic bath 218 is covered with a heat insulating material in order to keep the capillaries 202 in the thermostatic bath 218 at a constant temperature, and the temperature is controlled by a heating/cooling mechanism 220. Further, a fan 219 circulates and mixes the air in the thermostatic bath 218 to keep the temperature of the capillary array 217 positionally uniform and constant.

The conveyance device 225 conveys various containers to the capillary cathode ends 227. The conveyance device 225 includes three electric motors and a linear actuator, and is movable in three axial directions, namely, in the up and down direction, the left and right direction, and the depth direction. Further, at least one or more containers can be placed on a movable stage 230 of the conveyance device 225. The movable stage 230 has an electric grip 231 which can grip and release each container. Therefore, a buffer container 221, a cleaning container 222, a waste liquid container 223, and a sample plate 224 can be conveyed to the capillary cathode ends 227 as required. Containers which are not required are stored in a predetermined storage space in the electrophoresis device 110.

The user can use the data analyzer 111 to control various functions of the electrophoresis device 110 and to acquire electrophoresis data detected by the optical detection unit.

The electrophoresis device 110 may include sensors for acquiring information relating to an observation environment which affects electrophoresis (observation environment information). The electrophoresis device 110 of FIG. 2 includes internal sensors 240, polymer sensors 241, and a buffer sensor 242.

The internal sensors 240 are sensors for acquiring information relating to the internal environment of the electrophoresis device 110, and perform measurements by using, for example, a temperature sensor, a humidity sensor, and an atmospheric pressure sensor arranged in the electrophoresis device 110.

The polymer sensors 241 are sensors for acquiring information relating to the quality of the polymer, and are, for example, a PH sensor and an electrical conductivity sensor. In FIG. 2, the polymer sensors 241 are arranged in the polymer container 209, but the arrangement location is not limited thereto.

The buffer sensor 242 is a sensor for acquiring information relating to the quality of the buffer, and is, for example, a temperature sensor. In FIG. 2, the buffer sensor 242 is arranged in the anode buffer container 210, but the arrangement location is not limited thereto. For example, the buffer sensor 242 may be arranged in the buffer container 221.

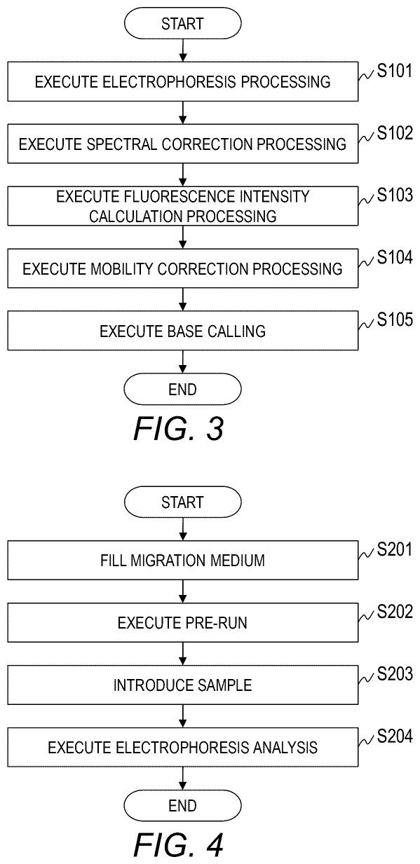

FIG. 3 is a flowchart for illustrating an outline of processing executed by the gene analyzer 100 of the first embodiment.

The electrophoresis device 110 of the gene analyzer 100 executes electrophoresis processing on a sample to be analyzed (Step S101). Details of the electrophoresis processing are described with reference to FIG. 4.

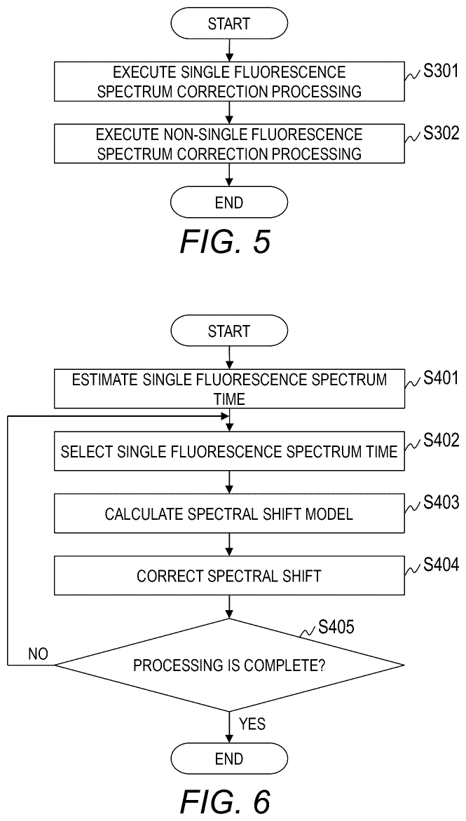

Next, the data analyzer 111 of the gene analyzer 100 executes spectral correction processing by using the electrophoresis data (Step S102). Details of the spectral correction processing are described with reference to FIG. 5.

Next, the data analyzer 111 of the gene analyzer 100 executes fluorescence intensity calculation processing by using the corrected electrophoresis data (Step S103). Specifically, the fluorescence intensity calculation module 133 calculates fluorescence intensity time-series data of a fluorescent dye from the corrected electrophoresis data, and detects the center position, height, and width, for example, of peaks from the fluorescence intensity time-series data.

Next, the data analyzer 111 of the gene analyzer 100 executes mobility correction processing on the fluorescence intensity time-series data (Step S104).

Next, the data analyzer 111 of the gene analyzer 100 executes base calling by using the fluorescence intensity time-series data corrected based on the results of the mobility correction processing (Step S105). Specifically, the base calling module 135 identifies the base sequence of the sample by using the corrected fluorescence intensity time-series data.

FIG. 4 is a flowchart for illustrating electrophoresis processing executed by the electrophoresis device 110 in the first embodiment.

The user sets samples to be analyzed and reagents, for example, in the electrophoresis device 110, and instructs the start of the electrophoresis processing via the coupling interface 122. The samples are set in accordance with the following steps.

The user fills the buffer container 221 and the anode buffer container 210 with a buffer for forming a part of a current-carrying path. The buffer is, for example, an electrolyte solution commercially available from various companies for use in electrophoresis. The user dispenses the samples to be analyzed into wells of the sample plate 224. The samples are, for example, a DNA PCR product. The user dispenses a cleaning solution for cleaning the capillary cathode end 227 into the cleaning container 222. The cleaning solution is, for example, pure water. The user injects into the syringe 206 a migration medium for electrophoresis the sample. Examples of the migration medium include polyacrylamide separation gels and polymers commercially available from various companies for use in electrophoresis. The user replaces the capillary array 217 when deterioration of the capillaries 202 is expected or to change the length of the capillaries 202.

At this time, examples of the samples to be set on the sample plate 224 include, in addition to the actual DNA sample to be analyzed, a positive control, a negative control, and an allelic ladder, and each is electrophoresed in a different capillary 202.

The positive control is, for example, a PCR product containing known DNA, and is a sample for a control experiment in order to confirm that the DNA is correctly amplified by PCR. The negative control is a PCR product that does not contain DNA, and is a sample for a control experiment in order to confirm that the PCR amplification product is free from contamination such as user DNA or dust. The allelic ladder is an artificial sample containing many bases that may be commonly included in DNA markers, and is usually provided by a reagent manufacturer as a reagent kit for DNA identification. The allelic ladder is used for the purpose of fine-tuning the correspondence between the DNA fragment length of individual DNA markers and alleles.

Further, a known DNA fragment, referred to as a size standard, labeled with a specific fluorescent dye is mixed in all of the actual sample, the positive control, the negative control, and the allelic ladder. The type of fluorescent dye assigned to the size standard differs depending on the reagent kit used.

The user designates, for example, the type of the allelic ladder, the type of the size standard, the type of the fluorescent reagent, and the type of each sample to be set in the wells on the sample plate 224 corresponding to each capillary 202. In the first embodiment, any one of the actual sample, the positive control, the negative control, and the allelic ladder is designated. Those information settings are input to the sample information setting module 131 via the coupling interface 122 of the data analyzer 111.

The above is description of the setting of the samples.

The electrophoresis device control module 132 transmits a signal instructing the start of analysis to the electrophoresis device 110. When the electrophoresis device 110 receives the signal, the electrophoresis device 110 starts the electrophoresis processing described below.

First, the electrophoresis device 110 fills new migration medium into the capillaries 202 to form a migration path (Step S201). The filling of the migration medium may be executed automatically after the start of analysis, or may be executed sequentially based on control signals transmitted from the electrophoresis device control module 132.

Specifically, the electrophoresis device 110 transports the waste liquid container 223 to directly below the load header 229 through use of the conveyance device 225, and closes an electromagnetic valve 213 to make it possible for the used migration medium discharged from the capillary cathode end 227 to be received. The electrophoresis device 110 then drives the syringe 206, fills the capillary 202 with new migration medium, and discards the used migration medium. Finally, the electrophoresis device 110 immerses the capillary cathode end 227 in the cleaning solution in the cleaning container 222 to clean the capillary cathode end 227 contaminated with the migration medium.

Next, the electrophoresis device 110 applies a predetermined voltage to the migration medium to execute a pre-run to bring the migration medium into a state suitable for electrophoresis (Step S202). The pre-run may be performed automatically, or may be performed sequentially based on control signals transmitted from the electrophoresis device control module 132.

Specifically, the electrophoresis device 110 uses the conveyance device 225 to immerse the capillary cathode end 227 into the buffer in the buffer container 221 to form a current-carrying path. Then, the electrophoresis device 110 applies a voltage of about several kilovolts to several tens of kilovolts to the migration medium for several minutes to several tens of minutes from the high-voltage power source 204 to bring the migration medium into a state suitable for electrophoresis. Finally, the electrophoresis device 110 immerses the capillary cathode end 227 in the cleaning solution in the cleaning container 222 to clean the capillary cathode ends 227 contaminated with the buffer.

Next, the electrophoresis device 110 introduces the sample (Step S203). The sample introduction may be performed automatically, or may be performed sequentially based on control signals sent from the electrophoresis device control module 132.

Specifically, the electrophoresis device 110 uses the conveyance device 225 to immerse the capillary cathode end 227 into the sample held in the well of the sample plate 224, and then opens the electromagnetic valve 213. This forms a current-carrying path, which makes it possible to introduce the sample components into the migration path. The electrophoresis device 110 applies a pulse voltage to the current-carrying path from the high-voltage power source 204 to introduce the sample components into the migration path. Finally, the electrophoresis device 110 immerses the capillary cathode end 227 in the cleaning solution in the cleaning container 222 to clean the capillary cathode end 227 contaminated by the sample.

Next, the electrophoresis device 110 executes electrophoresis analysis in which each sample component contained in the sample is separated and analyzed (Step S204). The electrophoresis analysis may be performed automatically, or may be performed sequentially based on control signals transmitted from the electrophoresis device control module 132.

Specifically, the electrophoresis device 110 uses the conveyance device 225 to immerse the capillary cathode end 227 into the buffer in the buffer container 221 to form a current-carrying path. The electrophoresis device 110 applies a high voltage of around 15 kilovolts to the current-carrying path from the high-voltage power source 204 to generate an electric field in the migration path. The generated electric field causes each sample component in the migration path to move to the detection unit 216 at a speed that depends on the properties of each sample component. That is, the sample components are separated based on the differences in speed at which each sample component moves. Then, the sample components which have reached the detection unit 216 are detected in order. For example, when the sample contains a large number of pieces of DNA having different base lengths, differences arise in the movement speed of the pieces of DNA depending on the base length, and hence the pieces of DNA having a shorter base length reach the detection unit 216 before the pieces of DNA having a longer base length. To each piece of DNA, a fluorescent dye which depends on its terminal base sequence is attached. When the detection unit 216 is irradiated with excitation light from the light source 214, fluorescence having a wavelength dependent on the sample is produced and emitted to the outside. The electrophoresis device 110 detects the fluorescence by using the optical detector 215. During electrophoresis analysis, the optical detector 215 detects this fluorescence at regular time intervals, and transmits image data to the data analyzer 111. It should be noted that in order to reduce the amount of information to be transmitted, in place of the image data, the luminance of only a part of areas of the image data may be transmitted. For example, for each capillary 202, a luminance value obtained through sampling only at wavelength positions at regular intervals may be transmitted. The data transmitted from the electrophoresis device 110 is time-series data of the luminance value of each capillary 202 (fluorescence intensity time-series data), and is stored in the storage device 121.

When the electrophoresis device 110 has acquired the expected image data, the electrophoresis device 110 stops applying a voltage and ends the electrophoresis analysis. The above is description of the electrophoresis processing.

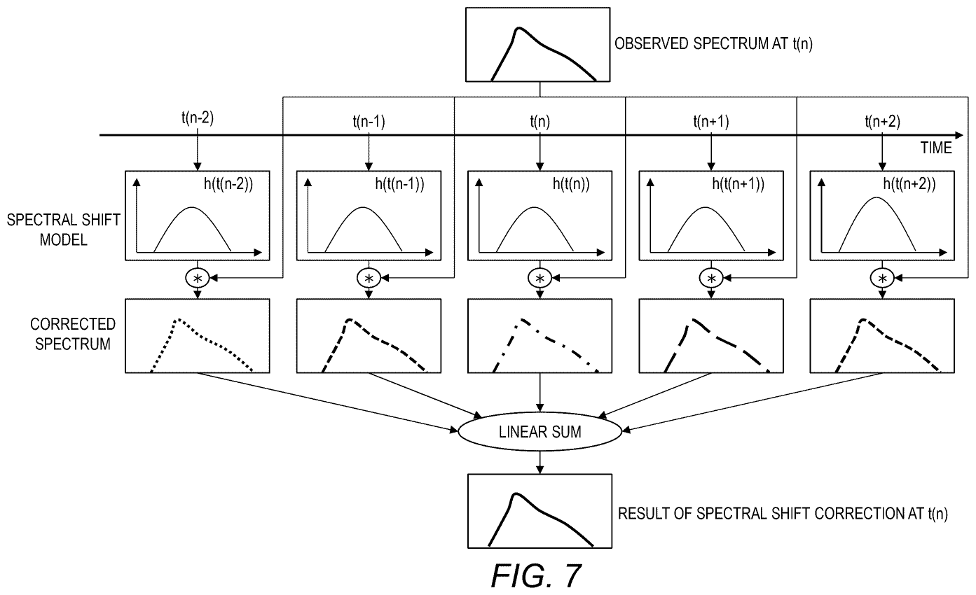

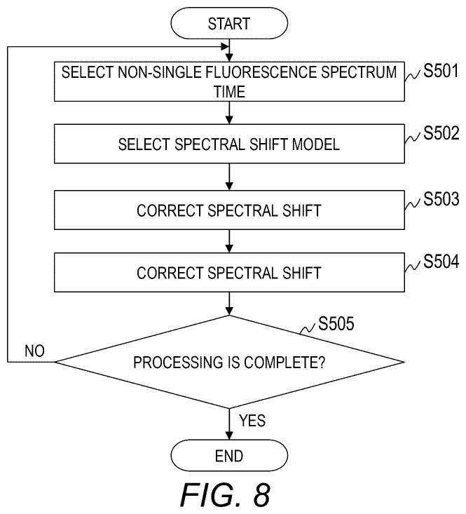

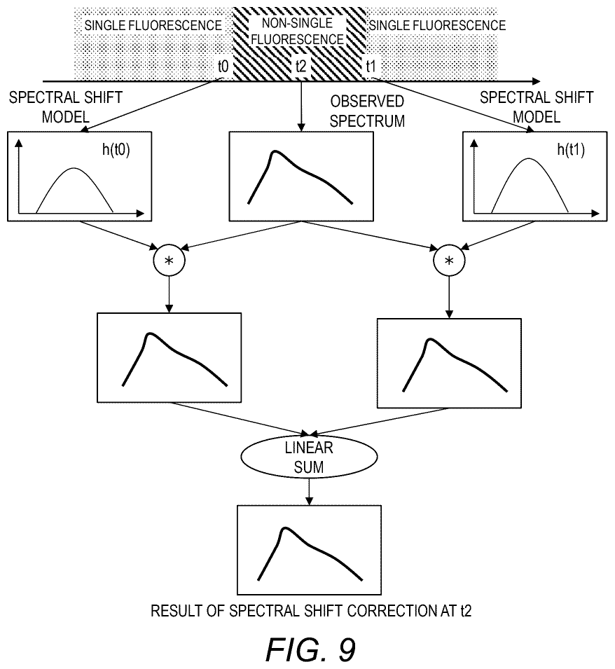

FIG. 5 is a flowchart for illustrating spectral correction processing executed by the data analyzer 111 in the first embodiment. FIG. 6 is a flowchart for illustrating single fluorescence spectrum correction processing executed by the data analyzer 111 in the first embodiment. FIG. 7 is a diagram for illustrating images of the single fluorescence spectrum correction processing in the first embodiment. FIG. 8 is a flowchart for illustrating non-single fluorescence spectrum correction processing executed by the data analyzer 111 in the first embodiment. FIG. 9 is a diagram for illustrating images of the non-single fluorescence spectrum correction processing in the first embodiment.

The spectral shift correction module 134 executes, on time-series data (electrophoresis data) of signal intensities of a plurality of frequencies, single fluorescence spectrum correction processing (Step S301), and then executes non-single fluorescence spectrum correction processing (Step S302).

As used herein, “single fluorescence spectrum” means a spectrum derived from only one base, and “non-single fluorescence spectrum” means a spectrum derived from a plurality of bases.

In the single fluorescence spectrum correction processing, the spectral shift correction module 134 uses the electrophoresis data to estimate a single fluorescence spectrum time (Step S401). As used herein, “single fluorescence spectrum time” means the time at which a single fluorescence spectrum is detected. Examples of the estimation method may include the following.

(Method 1) The spectral shift correction module 134 compares the spectrum at a time “t” and the spectrum obtained from a color conversion matrix. When the spectrum at the time “t” is similar to the spectrum obtained from the color conversion matrix, the spectral shift correction module 134 determines that the spectrum at the time “t” is a single fluorescence spectrum, and records the time “t” as the single fluorescence spectrum time. The color conversion matrix is a matrix in which the elements are coefficients for obtaining individual fluorescence intensities from a spectral waveform. The color conversion matrix may be a color conversion matrix obtained in advance by using a reagent referred to as a “matrix standard,” in which DNA fragments of different lengths are each labeled with a different fluorescent dye, for use during electrophoresis (for example, see JP 2014-117222 A). In addition, a color conversion matrix may be obtained each time a sample is run (for example, see JP 2020-41876 A).

(Method 2) The spectral shift correction module 134 calculates a fluorescence intensity vector by applying a color conversion matrix to the spectrum (n-th order vector) at a time “t”. When a pull-up component is equal to or less than a threshold value, that is, when a value other than the value of a given base component of the fluorescence intensity vector is equal to or less than a threshold value, the spectral shift correction module 134 determines that the spectrum at the time “t” is a single fluorescence spectrum, and records the time “t” as the single fluorescence spectrum time.

Method 1 and Method 2 may be combined. Further, a method in which an estimation is performed by using, for example, a difference in impulse response waveforms or an FFT characteristic of a wavelength spectrum can also be used.

The spectral shift correction module 134 selects one single fluorescence spectrum time from among the single fluorescence spectrum times estimated in Step S401 (Step S402). Here, it is assumed that the selection is performed in chronological order.

The spectral shift correction module 134 calculates a spectral shift model at the single fluorescence spectrum time (Step S403). Specifically, the spectral shift correction module 134 calculates a spectral shift model by using Expression (1). In Expression 1, ω represents the frequency, F₀(ω) represents the Fourier transform of the spectrum obtained from the color conversion matrix, F₁(ω) represents the Fourier transform of the single fluorescence spectrum, and H_{M}(ω) represents the spectral shift model.

$${H}_{M} ( ω ) = \frac{{F}_{1} ( ω )}{{F}_{0} ( ω )} \qquad ( 1 )$$

In this embodiment, the single fluorescence spectrum is regarded as a shifted spectrum obtained from the color conversion matrix, and an impulse response for correcting this shift is calculated as the spectral shift model.

The spectral shift correction module 134 corrects the spectral shift of the single fluorescence spectrum by using the spectral shift model (Step S404). Specifically, the spectral shift correction module 134 corrects the spectral shift of the single fluorescence spectrum by calculating H_{M}(t) by performing an inverse Fourier transform on H_{M}(ω) and convolving H_{M}(t) with the single fluorescence spectrum F(t).

The spectral shift correction module 134 determines whether or not the processing for all estimated single fluorescence spectrum times is complete (Step S405).

When the processing for all estimated single fluorescence spectrum times is not complete, the spectral shift correction module 134 returns the process to Step S402, and executes the same processing.

When the processing for all estimated single fluorescence spectrum times is complete, the spectral shift correction module 134 ends the single fluorescence spectrum correction processing.

The single fluorescence spectrum correction processing illustrated in FIG. 6 is an example, and this invention is not limited to this. For example, a correction method as illustrated in FIG. 7 may be performed.

(Procedural Step 1) The spectral shift correction module 134 calculates a spectral shift model for each single fluorescence spectrum time.

(Procedural Step 2) The spectral shift correction module 134 selects a single fluorescence spectrum time t(n), and acquires a spectral shift model for the single fluorescence spectrum time t(n) and each of the preceding and following single fluorescence spectrum times. It should also be noted that the spectral shift models of a predetermined number of single fluorescence spectrum times (a predetermined time range) before and after the single fluorescence spectrum time “t” may be acquired.

(Procedural Step 3) The spectral shift correction module 134 calculates a plurality of correction spectra by convolving each spectral shift model with the single fluorescence spectrum F(t(n)) at the single fluorescence spectrum time t(n).

(Procedural Step 4) The spectral shift correction module 134 outputs the linear sum of the plurality of correction spectra as the final correction result.

The above is description of the single fluorescence spectrum correction processing.

In the non-single fluorescence spectrum correction processing, the spectral shift correction module 134 selects a non-single fluorescence spectrum time (Step S501). The non-single fluorescence spectrum time is a time other than the single fluorescence spectrum time estimated in Step S401. Here, it is assumed that the selections are made in chronological order.

The spectral shift correction module 134 selects a spectral shift model to be used from among the spectral shift models calculated in the single fluorescence spectrum correction processing (Step S502).

Specifically, as illustrated in FIG. 9, the spectral shift correction module 134 acquires the spectral shift model of each of a single fluorescence spectrum times t0 and t1 before and after a non-single fluorescence spectrum time t2. It should also be noted that the spectral shift model of a predetermined number of single fluorescence spectrum times before and after a non-single fluorescence spectrum time may be acquired.

The spectral shift correction module 134 calculates a plurality of correction spectra by convolving each spectral shift model with the non-single fluorescence spectrum at the non-single fluorescence spectrum time (Step S503).

The spectral shift correction module 134 acquires the linear sum of the plurality of correction spectra as the final correction result (Step S504).

The spectral shift correction module 134 determines whether or not processing for all non-single fluorescence spectrum times is complete (Step S505).

When the processing for all non-single fluorescence spectrum times is not complete, the spectral shift correction module 134 returns the process to Step S501, and executes the same processing.

When the processing for all non-single fluorescence spectrum times is complete, the spectral shift correction module 134 ends the non-single fluorescence spectrum correction processing.

Whether or not the spectral correction processing in this embodiment is functioning effectively can be confirmed by the following method. Migration data in which non-single fluorescence spectra are present in a wide electrophoresis time band at a fixed mixed ratio and a single fluorescence spectrum is present in a part of the time band is input to the spectral shift correction module 134. When the mixed ratio between the non-single fluorescence spectra near a single fluorescence spectrum and other non-single fluorescence spectra is significantly different among the non-single fluorescence spectra obtained from the spectral shift correction module 134, this indicates that the spectral correction processing in this embodiment is functioning effectively.

According to this embodiment, pull-ups caused by spectral shift can be reduced through information processing. As a result, the accuracy of base sequence analysis can be improved. This embodiment also has a feature of having high versatility because this embodiment does not depend on the implementation of the electrophoresis device 110.

The present invention is not limited to the above embodiment and includes various modification examples. In addition, for example, the configurations of the above embodiment are described in detail so as to describe the present invention comprehensibly. The present invention is not necessarily limited to the embodiment that is provided with all of the configurations described. In addition, a part of each configuration of the embodiment may be removed, substituted, or added to other configurations.

A part or the entirety of each of the above configurations, functions, processing units, processing means, and the like may be realized by hardware, such as by designing integrated circuits therefor. In addition, the present invention can be realized by program codes of software that realizes the functions of the embodiment. In this case, a storage medium on which the program codes are recorded is provided to a computer, and a CPU that the computer is provided with reads the program codes stored on the storage medium. In this case, the program codes read from the storage medium realize the functions of the above embodiment, and the program codes and the storage medium storing the program codes constitute the present invention.

Examples of such a storage medium used for supplying program codes include a flexible disk, a CD-ROM, a DVD-ROM, a hard disk, a solid state drive (SSD), an optical disc, a magneto-optical disc, a CD-R, a magnetic tape, a non-volatile memory card, and a ROM.

The program codes that realize the functions written in the present embodiment can be implemented by a wide range of programming and scripting languages such as assembler, C/C++, Perl, shell scripts, PHP, Python and Java.

It may also be possible that the program codes of the software that realizes the functions of the embodiment are stored on storing means such as a hard disk or a memory of the computer or on a storage medium such as a CD-RW or a CD-R by distributing the program codes through a network and that the CPU that the computer is provided with reads and executes the program codes stored on the storing means or on the storage medium.

In the above embodiment, only control lines and information lines that are considered as necessary for description are illustrated, and all the control lines and information lines of a product are not necessarily illustrated. All of the configurations of the embodiment may be connected to each other.

What is claimed is:

1. A base sequence analysis method, which is executed by a gene analyzer configured to analyze a base sequence of a sample, the base sequence analysis method comprising:
a first step of acquiring, by the gene analyzer, electrophoresis data which is time-series data of signal intensities of a plurality of frequencies acquired by subjecting the sample to electrophoresis;
a second step of identifying, by the gene analyzer, a single fluorescence spectrum time at which a single fluorescence spectrum which is a spectrum derived from only one base is present, by using the electrophoresis data;
a third step of calculating, by the gene analyzer, a spectral shift model by using a spectrum obtained from a color conversion matrix at the single fluorescence spectrum time and the single fluorescence spectrum at the single fluorescence spectrum time;
a fourth step of correcting, by the gene analyzer, the electrophoresis data by using the spectral shift model; and
a fifth step of identifying, by the gene analyzer, a base sequence of the sample by using the corrected electrophoresis data.

2. The base sequence analysis method according to claim 1,
wherein the spectral shift model is a function of a frequency space, and
wherein the fourth step includes a step of correcting, by the gene analyzer, a spectrum at a given time by executing a convolution operation using the spectrum at the given time and the spectral shift model.

3. The base sequence analysis method according to claim 2, wherein the fourth step includes:
a sixth step of correcting, by the gene analyzer, the single fluorescence spectrum at the single fluorescence spectrum time by using the spectral shift model at the single fluorescence spectrum time; and
a seventh step of correcting, by the gene analyzer, at a time other than the single fluorescence spectrum time, a spectrum at the time by using the spectral shift model at at least one single fluorescence spectrum time near the time.

4. The base sequence analysis method according to claim 3, wherein the sixth step includes the steps of:
selecting, by the gene analyzer, a first single fluorescence spectrum time and acquiring the spectral shift model at the first single fluorescence spectrum time;
acquiring, by the gene analyzer, the spectral shift model at another single fluorescence spectrum time included in a predetermined time range including the first single fluorescence spectrum time; and
correcting the single fluorescence spectrum at the first single fluorescence spectrum time by using a plurality of the spectral shift models.

5. A gene analyzer for analyzing a base sequence of a sample, the gene analyzer comprising:
a calculation device; and
a storage device coupled to the calculation device;
the calculation device being configured to:
acquire electrophoresis data which is time-series data of signal intensities of a plurality of frequencies acquired by subjecting the sample to electrophoresis;
identify a single fluorescence spectrum time at which a single fluorescence spectrum which is a spectrum derived from only one base is present, by using the electrophoresis data;
calculate a spectral shift model by using a spectrum obtained from a color conversion matrix at the single fluorescence spectrum time and the single fluorescence spectrum at the single fluorescence spectrum time;
correct the electrophoresis data by using the spectral shift model; and
identify a base sequence of the sample by using the corrected electrophoresis data and output an analysis result of the base sequence of the sample.

6. The gene analyzer according to claim 5,
wherein the spectral shift model is a function of a frequency space, and
wherein the calculation device is configured to correct a spectrum at a given time by executing a convolution operation using the spectrum at the given time and the spectral shift model.

7. The gene analyzer according to claim 6, wherein the calculation device is configured to execute:
first correction processing of correcting the single fluorescence spectrum at the single fluorescence spectrum time by using the spectral shift model at the single fluorescence spectrum time; and
second correction processing of correcting, at a time other than the single fluorescence spectrum time, a spectrum at the time by using the spectral shift model at at least one single fluorescence spectrum time near the time.

8. The gene analyzer according to claim 7, wherein the calculation device is configured to, in the first correction processing:
select a first single fluorescence spectrum time and acquire the spectral shift model at the first single fluorescence spectrum time;
acquire the spectral shift model at another single fluorescence spectrum time included in a predetermined time range including the first single fluorescence spectrum time; and
correct the single fluorescence spectrum at the first single fluorescence spectrum time by using a plurality of the spectral shift models.
